(19) United States
(12) Patent Application Publication
(10) Pub. No.: US 2025/0145678 A1
(43) Pub. Date: May 8, 2025

(54) METHOD OF USING/APPLYING A KERATIN HYDROLYSIS PEPTIDE SOLUTION TO ENHANCE THE DROUGHT TOLERANCE ATTRIBUTE OF WHEAT PLANTS

(71) Applicant: CH Biotech R&D Co., Ltd., Nantou City (TW)

(72) Inventors: Kai XIA, Nantou (TW); Cheng-Ku LIN, Taichung City (TW); Ya-Ling LIN, Changhua (TW)

(73) Assignee: CH Biotech R&D Co., Ltd., Nantou City (TW)

(21) Appl. No.: 18/408,257

(22) Filed: Jan. 9, 2024

(30) Foreign Application Priority Data
Nov. 3, 2023 (TW) 112142536

(51) Int. Cl. C07K 14/47; A01N 37/46; A01P 21/00; C05F 1/00

(52) U.S. Cl. CPC C07K 14/4741; A01N 37/46; A01P 21/00; C05F 1/005

(57) ABSTRACT

Present invention teaches the method of using a keratin hydrolysis peptide (“KHP”) solution to enhance the drought tolerance attribute of wheat plants. By selectively choosing specific weights of feathers and water, and treating the mixture to a high-temperature high-pressure hydrolysis process, the resulting solution is confirmed to contain at least 253 peptides and then infused to the soil in which the wheat seeds are planted; the solution can also be sprayed to the leaf surface of young wheat plants. Optionally, the KHP solution can be diluted by water, as disclosed in the specification, for applying to the soil around the wheat plants and for spraying to leaf surface of young wheat plants.

### PRIORITY CLAIM TO FOREIGN APPLICATION

Applicant hereby makes priority claim to a Taiwan application, number 112142536, having the Taiwan filing date of Nov. 3, 2023.

### BACKGROUND OF THE INVENTION

Present invention disclosed and claimed the method and application of a keratin hydrolysis peptide (“KHP”) solution for enhancing the drought tolerance attribute of wheat plants, by using the thermally hydrolyzed feather keratin peptide solution to the soil when the wheat seeds are sowed as well as to the leave's surface of young wheat plants.

The KHP solution is made by a hydrolysis process using feathers and water, with one version of the solution using feathers only, via a high-temperature and high-pressure process, resulting in a solution that has many beneficial applications in the fields of horticulture, agriculture and potentially other farming businesses.

Hydrolyzed keratin has long been used to strengthen hairs, reduce hair splitting and breakage. Other beneficial uses include skin moisturization and wound healing. Keratin hydrolysate has also been known to function as a biofertilizer, boosting plants' growth by enhancing the plants' ability to receive and utilize nutrients, including commonly applied fertilizers.

The common wheat plant (Triticum aestimum L.), a species of Poaceae, is among the three primary grain crops (corn, rice and wheat) that are widely planted around the world and consumed by humans as staple food items for centuries. In addition to serving as human food, the wheat crop is also used to produce animal feeds.

In the United States, the wheat production ranks among the top three primary farming crops in recent years. People put a lot of resources into making sure that the growth and harvesting of wheat would be sufficient for both domestic and exportation.

The onslaught of global weather change has caused severe strain on the production capacity of wheat, in addition to other environment issues, such as water shortage, overuse of chemical fertilizers, etc. Among the production obstacles, wheat production is subject to severe negative impact due to droughts. Studies have shown that the seasonal and unexpected droughts have caused no less than 21% production loss annually.

Studies further show that, given the temperature rise of 1° C., the global production of wheat may be reduced by 4.1-6.4%, based upon World Agricultural Production Bulletin issued by U.S. Department of Agriculture. The drought caused by the higher temperature has become an unavoidable problem that people have to deal with without further delay.

The lack of water brought on by drought may cause slowed growth, at the minimum, and would completely destroy the wheat production for the whole growth season if the drought is extreme. The amount of chlorophyll in wheat plants will be reduced, causing the photosynthesis function to be degraded, in a drought condition.

As such, people have put in substantial research and efforts to counter the adverse conditions caused by drought. Several patented methods and substances have disclosed and claimed different ways/substances of countering the wheat's drought adversities. The claimed remedies include, but not limited to, some genetic modification, chemical based nutrition supplements, specific fertilizers, etc. However, all the known methods/techniques are limited by their inherent technicalities: the complexity of the process and the application to specific wheat species. Put simply, these existing methods are not cost-effective for using to a wide spectrum of wheat species.

The present invention's KHP solution, on the contrary, is not limited by specific wheat species and is way more cost-effective to implement, with very low environmental impact. The inventor's field tests, as discussed herein, confirmed the effectiveness of applying the KHP solution to enhance the drought tolerance as disclosed herein.

### SUMMARY OF THE INVENTION

The keratin solution is primarily based upon feather, which contains 85-91% keratin, 13-15% organic nitrogen,1.6-2% organic sulfur, as well as other materials. The high keratin content has drawn many prior researches that work to break down, by enzyme, chemical agents, or fermentation process, into peptides, amino acids and other smaller molecules that can be used for animal feeds, plant fertilizers, and cultivation bases.

Around 2019, Nurdiawati, et al, came up with a hydrolysis process, by the mixture of α-amylase and protease to hydrolyze feather waste, resulting in a mixture of amino acids, fatty acids, and sugars. Nurdiawati experimented and adopted certain specific high-temperature and high-pressure setting in the hydrolysis process and discovered that the resulting solution, when mixed with some potassium and other minerals, can boost the growth of Pogostemon cablin and Vigna radiata, as reported in International Journal of Recycling or Organic Waste in Agriculture (8:221-232, 2019).

In 2016, Mr. Chung-Wu Dao, et al, disclosed a method to acidify feather powders as protein additives for organic composting fertilizers, boosting the absorption and utilization of fertilizers on account of organic fermentation. However, such process requires a proper acid-base neutralization adjustment, with potential damages of the acid or base liquids' leakage and ultimate disposal, resulting in additional post-processing costs. The selection of feather powders is, in and of itself, another complicated issues that need to have matching microbial agents for the acidification to work efficiently. There is no sufficient knowledge based on all the pro-and-con spectrum of uses that can provide a good and cost-effective guidance for the peptide hydrolysis method herein.

The inventors of present application, under the aegis of CH Biotech, developed and selected different feather and water compositions to perform the hydrolysis at higher temperature and higher pressure setting, resulting with different keratin hydrolysis peptide (“KHP”) solution that can be used on different crops/plants.

The selected embodiment of present invention uses a mixture of water and feathers (water content 50%) at the weight ratio of 5:4 (specific weight of 50 kg of feathers and 40 kg of water) and subject the mixture to a thermal hydrolysis process (80 minutes) to create a solution, resulting in the solution having molecular mass of 593.3-3,508.9 Dalton and containing at least 253 peptides as confirmed by using a mass spectrometer.

The inventors used the spectrometer Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

The solution then is applied to the soil in which the wheat seeds are planted.

The solution can be diluted by water, at 50 to 1000 ratio by volume, and then applied to the soil in which the wheat seeds are planted and to the leaf surface of young wheat plants.

### BRIEF DESCRIPTION OF DRAWINGS

The accompanying drawings, figures and tables, which are incorporated in and constitute a part of this specification, illustrate and exemplify the preferred embodiments of the invention. Together with the description, serve to explain the principles of the invention.

Table I shows the at least 253 peptides and its annotated sequences for the solution generated in accordance with the disclosure of this application.

FIG. 1A shows the comparison of above-ground dry weight of the biomass between the CK group and KHP-500x group, showing the enhanced drought tolerance exhibited by the wheat plants sprayed with the KHP solution.

FIG. 1B shows the comparison of the SPAD chlorophyll count between the CK group and the KHP-500x group.

FIG. 2A shows the above-ground stem width comparison among the 4 groups: normal group, check (CK) group, KHP-100x and KHP-50x. The drought tolerance of the two groups is substantially better than the check group when experiencing the same level of drought.

FIG. 2B shows the above-ground leaf length comparison among the 4 groups.

FIG. 2C shows the above-ground side shoot tillering comparison among the 4 groups.

FIG. 2D shows the above-ground biomass dry weight comparison among the 4 groups.

### DETAILED DESCRIPTION OF THE INVENTION

The keratin hydrolysis peptide (“KHP”) solution of present invention is made by a high-temperature and high-pressure process to treat a mixture of water and feathers as shown in the parameters herein.

The hydrolysis process takes the steps of:
a. Preparing the KHP solution by mixing 50 kg of feathers whose content is 50% water with 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm.

The confirmation of some of the bioactive 253 peptides is further done by referencing the BIOPEP-UWM database.

The solution can then be applied to the soil in which the wheat seeds are planted, as well as spraying to the leaf surface of the young wheat plants.

In addition to the specific implementation of 50 kg feathers and 40 kg water, the KHP solution can be made by another 2 sets of parameters shown herein:

| | | | Water | | | | | |
|---|---|---|---|---|---|---|---|---|
| | | | Content | | | | | |
| | Feather | Water | in | | | | | |
| | Weight | Weight | Feathers | Presure | Temp | Time | Mass | Concentr. |
| | (kg) | (kg) | % | (kg/cm²) | (° C.) | (min) | (Dalton) | (ppm) |
| | 66 | 44 | 50% | 16 | 195 | 40 | 593.3~3828.0 | 200000 |
| KHP | 50 | 40 | 50% | 12 | 185 | 80 | 593.3~3508.9 | 301500 |
| implementation | | | | | | | | |
| selected | | | | | | | | |
| | 70 | 0 | 46% | 13 | 180 | 40 | 705.9~3194.7 | 381250 |

The method of using a keratin hydrolysis peptide (KHP) solution stated above further causes the solution to be diluted with water by volume at the ratio of 1:50-1000 for applying to the soil in which the wheat seeds are planted, or spraying to the leaf's surface of the young wheat plants.

To test the effectiveness of the KHP solution made by the steps disclosed herein, the inventors chose the second embodiment (having 50 kg feathers and 40 kg water) solution to conduct the field tests as further described herein. This embodiment solution will be referred to as the KHP solution.

Additionally, during the field tests, then inventors selected different water dilution ratios, by volume, at 1:50, or 1:100, or 1:500, to compare the result of drought tolerance performance. The dilutions will be simply noted as 50×, 100×, or 500x to the KHP solution used.

The field tests consist of five (5) groups of wheat plants in normal grown pots with the growth condition of day time temperature 25° C., night time temperature 23° C.; day light hours of 16 and night time hours of 8. The day time light strength is 35000 lux.

The 5 groups are: normal group, check (CK) group, KHP-50x, KHP-100x and KHP-500x. The tests are done at different stages: seeding stage (KHP solution infused into the soil) and young plants (KHP solution sprayed to leaf surface).

For the KHP-500x comparison, water was given to the wheat seeds in the CK group and the KHP-500x regularly for 15 days, then the KHP solution was sprayed to the leaf surface of the KHP-500x group. For the next eight (8) days, no water was given to the two groups. As the signs of withering start to show, 3 more days' of regular water was given to both groups.

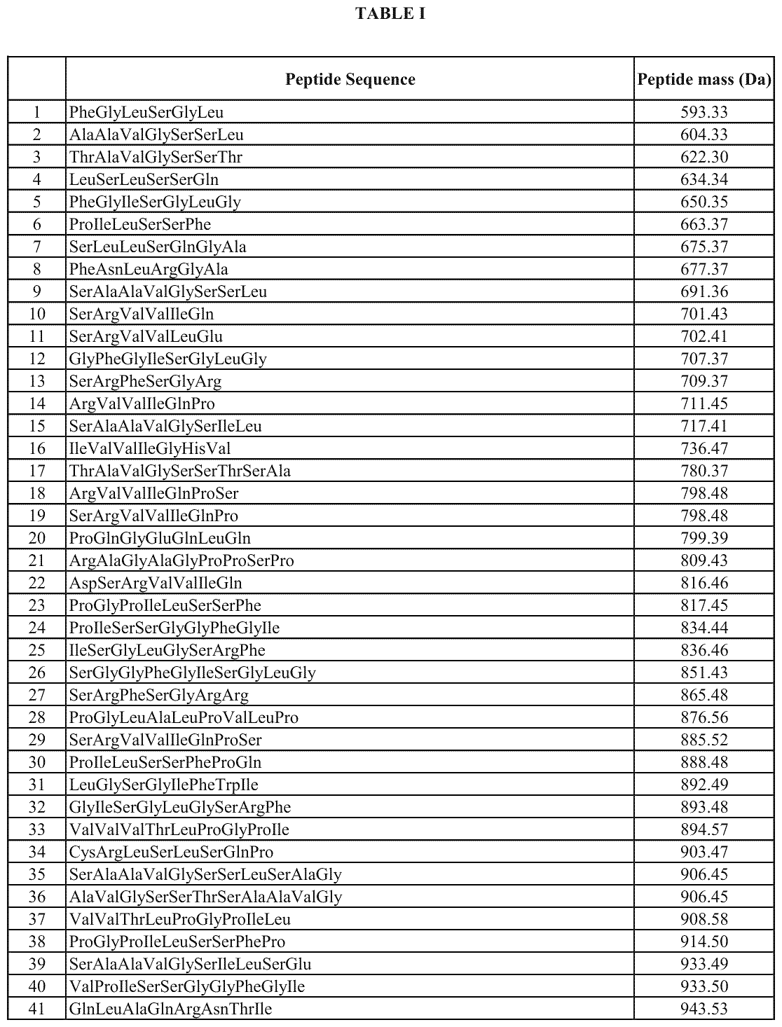

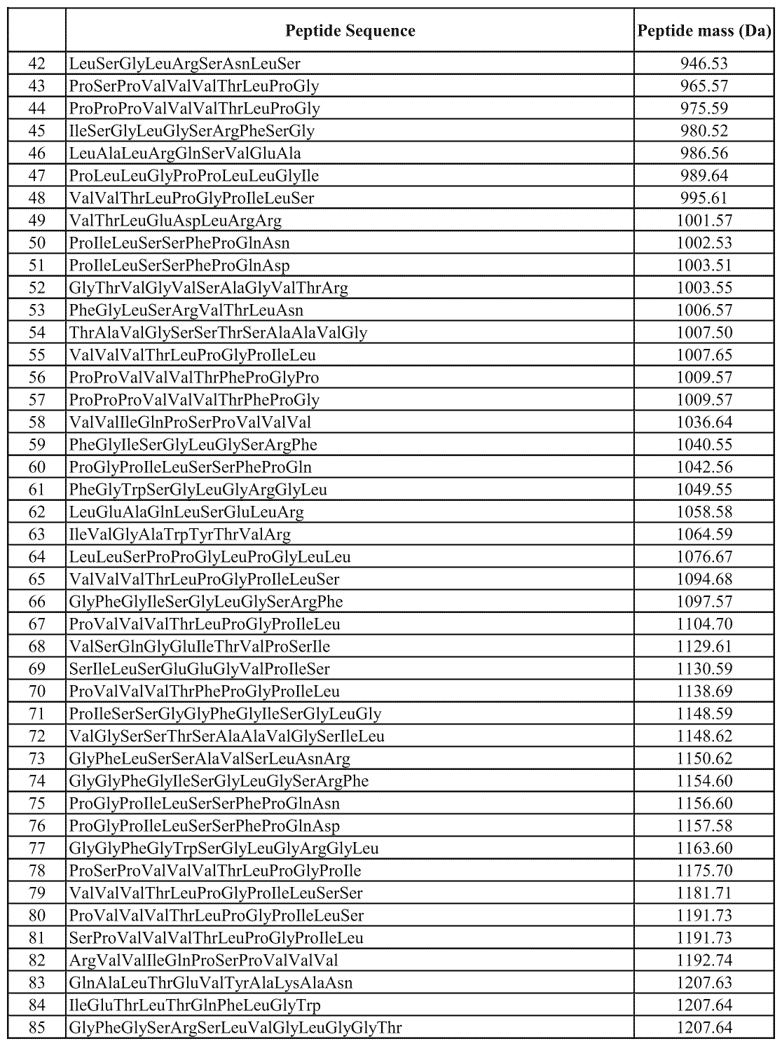

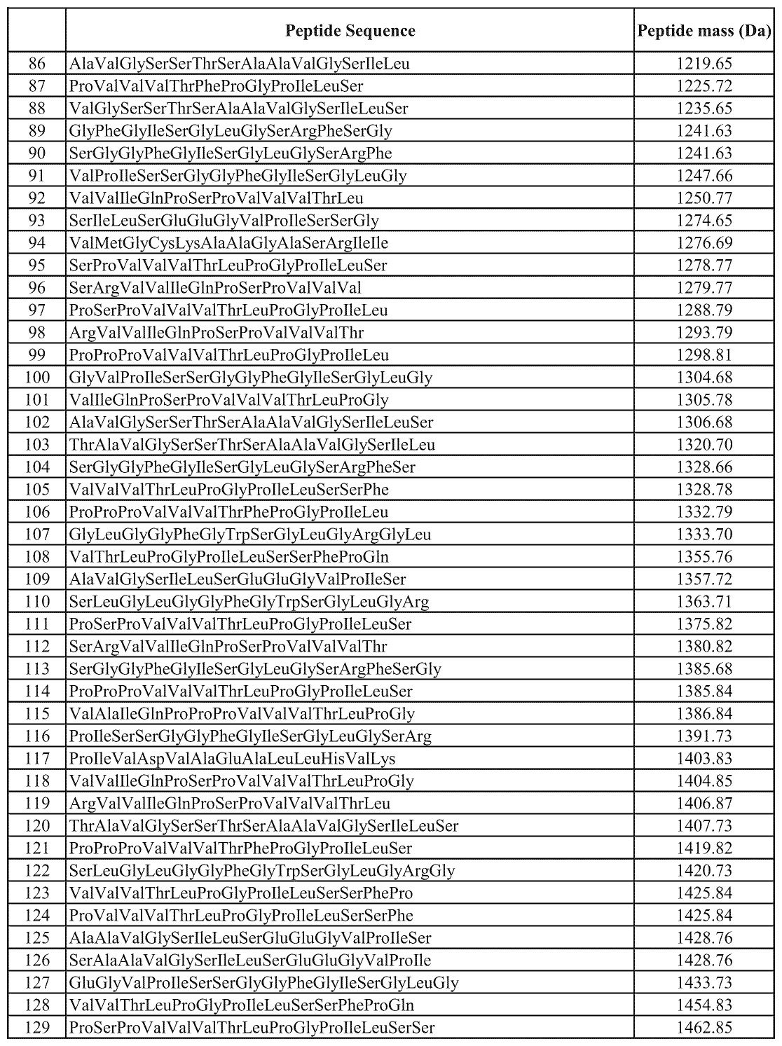

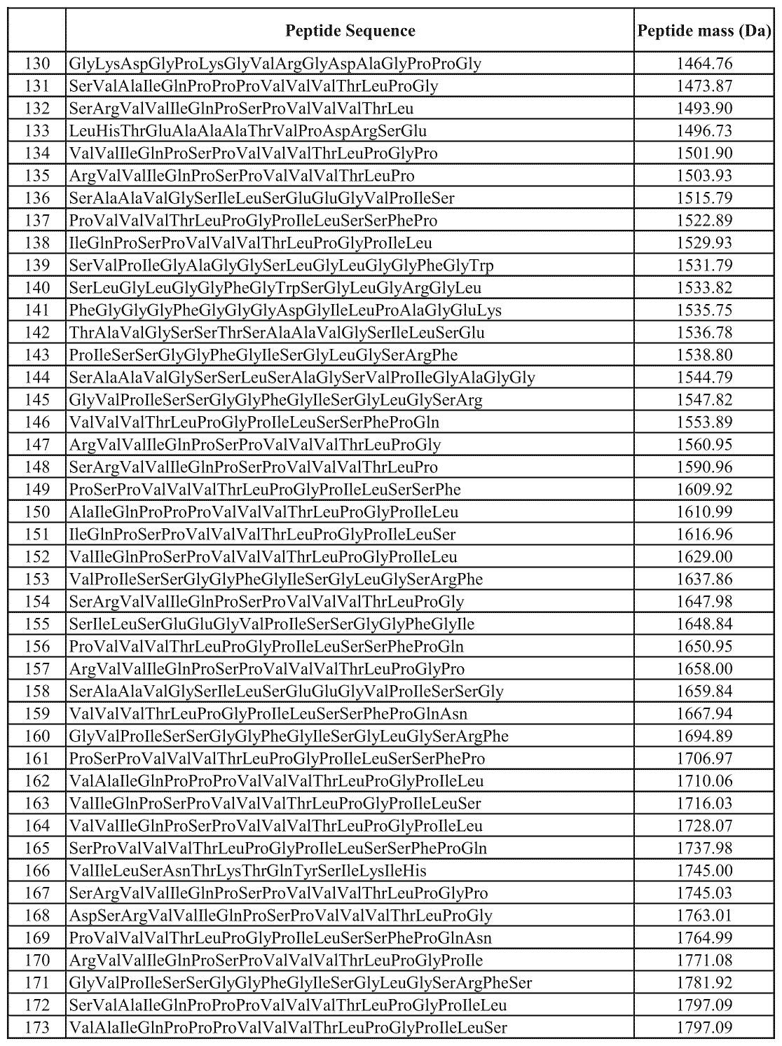

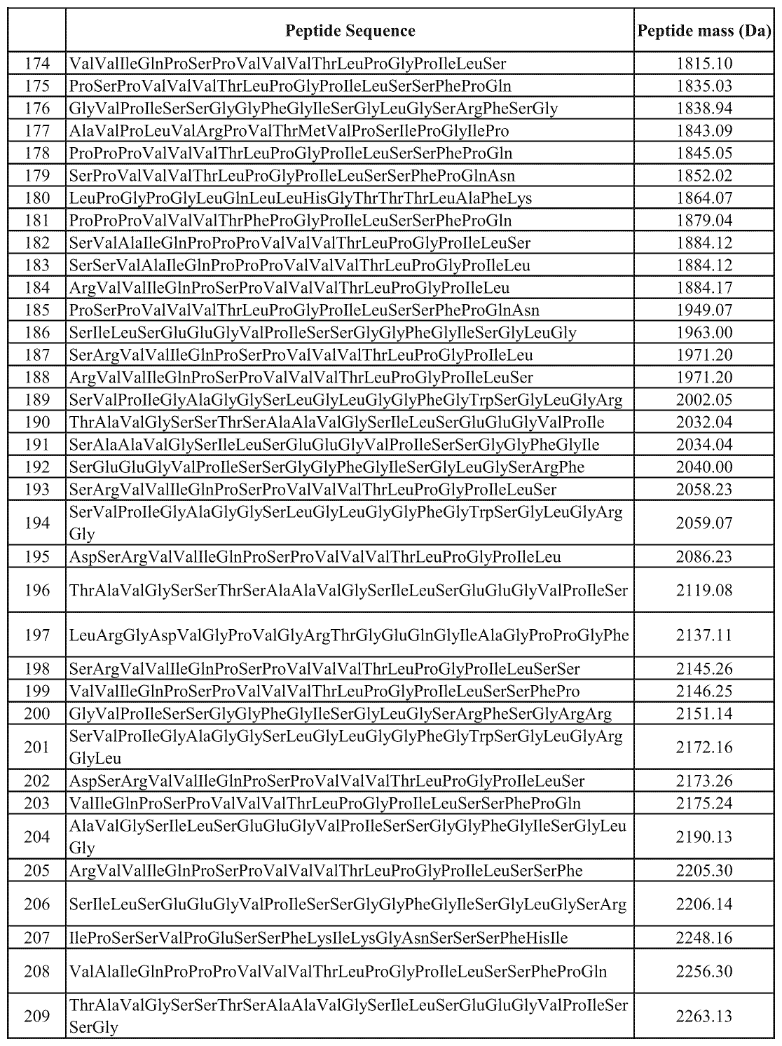

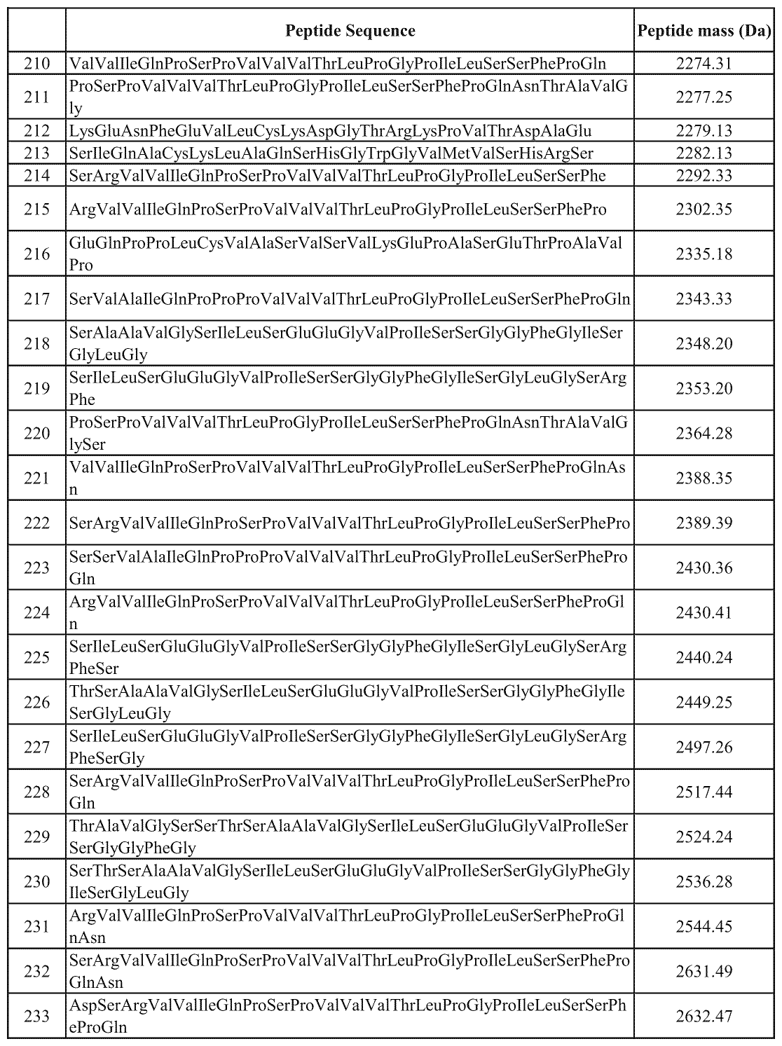

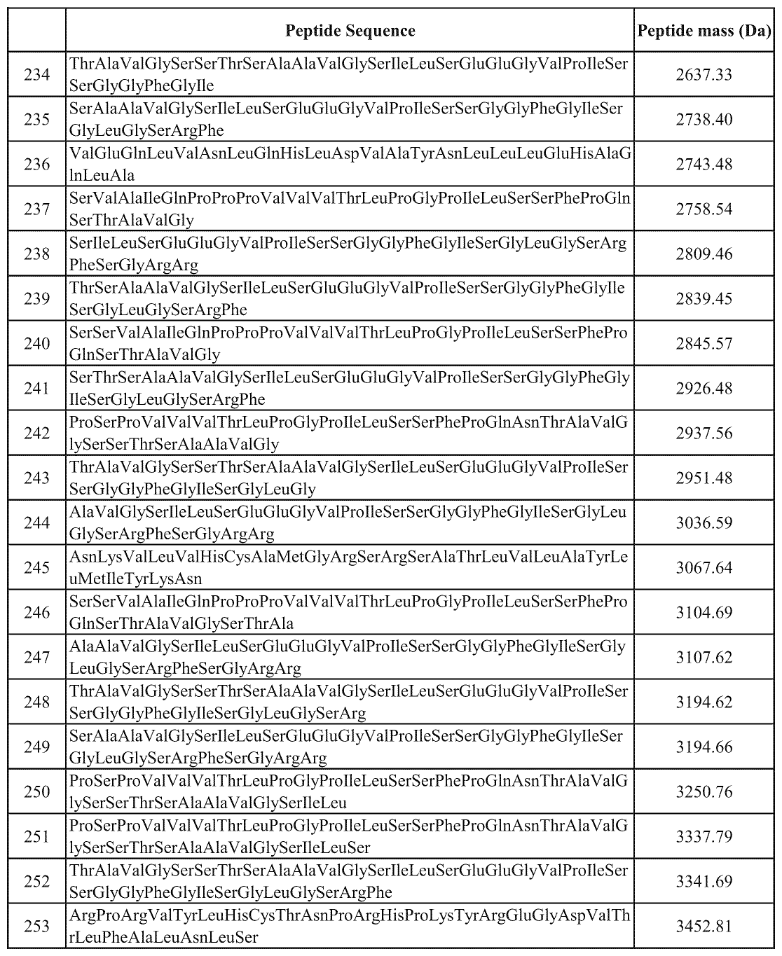

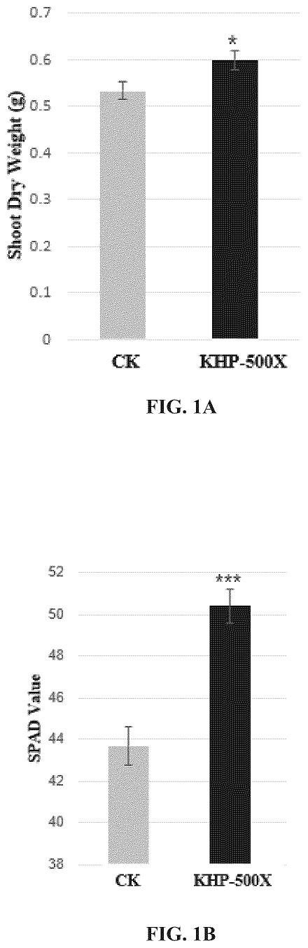

The above-ground biomass from the two groups are taken and weighed, after drying, on a digital scale (AP224X, Shimadazu). The comparison result is noted in FIG. 1A.

The same amount of full-grown leaves are taken from the two groups and are subject to a SPAD chlorophyll reading (2900PDL, Spectrum Technologies). The comparison result is noted in FIG. 1B.

As can be seen clearly, after suffering a period of drought, the KHP-500x group shows a stronger tolerance than the CK group, shown in FIGS. 1A and 1B.

For the KHP-50x and KHP-100x comparisons, regular amount of water is given to all groups at initial planting. Then check group and the 2 KHP groups are no longer given water for 15 days, followed by 3 days of regular watering. The normal group's watering remained unchanged.

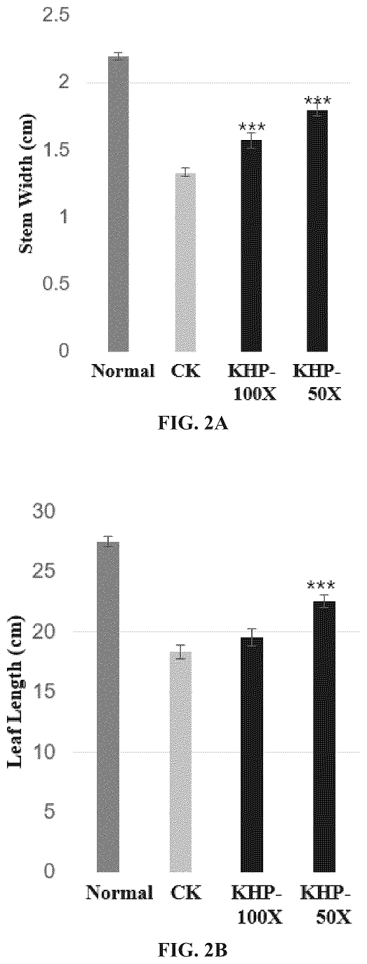

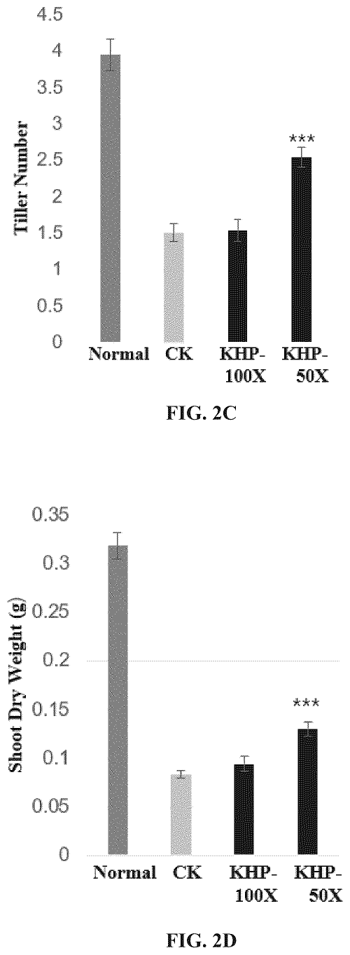

FIG. 2A shows that stem width comparison among the 4 groups. The 2 KHP groups, when given the KHP solutions (50× and 100×) show stronger drought tolerance than the CK group.

For leaf length measurement (FIG. 2B), offshoot tillering (FIG. 2C) and dry weight, the 2 KHP groups clearly perform much better drought tolerance than the CK group.

As has been proven by the field tests, and the scientific analysis/measurement done by the inventors, the method of creating the KHP solution and the method of application to wheat crops will help with enhancing the drought tolerance of the wheat plants.

While the disclosure herein gave limited teachings and embodiment examples, it should be noted that the description and disclosure made herein illustrated the preferred embodiments of the invention and are not meant to limit the scope of the applicant's rights. Variations and alterations may be employed for yet additional embodiments without departing from the scope of the invention herein.

1. A method of using a keratin hydrolysis peptide (KHP) solution to enhance the drought tolerance for wheat plants, comprising the steps of:
a. Preparing the KHP solution by mixing 50 kg of feathers whose content is 50% water and 40 kg of water in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to soil in which the wheat seeds are planted.

2. The method of using a keratin hydrolysis peptide (KHP) solution of claim 1 where the solution is sprayed to young wheat plant's leaf surfaces.

3. The method of using a keratin hydrolysis peptide (KHP) solution of claim 1 wherein the solution is diluted with water by volume at the ratio of 1:50-1000 for applying to the soil in which the wheat seeds are planted.

4. The method of using a keratin hydrolysis peptide (KHP) solution of claim 2 wherein the solution is diluted with water by volume at the ratio of 1:50-1000 for spraying to young wheat plant's leaf surfaces.
